(19) United States
(12) Patent Application Publication
(10) Pub. No.: US 2024/0079227 A1
(43) Pub. Date: Mar. 7, 2024

(54) HAIR REMOVAL INSTRUMENT

(71) Applicant: Shenzhen Ulike Smart Electronics Co., Ltd., Shenzhen (CN)

(72) Inventor: Shaoqing FANG, Shenzhen (CN)

(73) Assignee: Shenzhen Ulike Smart Electronics Co., Ltd., Shenzhen (CN)

(21) Appl. No.: 18/506,260

(22) Filed: Nov. 10, 2023

Related U.S. Application Data
(63) Continuation of application No. PCT/CN2023/101524, filed on Jun. 20, 2023.

(30) Foreign Application Priority Data
Jun. 20, 2022 (CN) 202221550413.3
Jun. 20, 2022 (CN) 202221551331.0
Jun. 20, 2022 (CN) 202221551332.5

(51) Int. Cl. H01J 61/52; A61B 18/20; H01J 61/02; H01J 65/00; H05K 7/20

(52) U.S. Cl. CPC H01J 61/52; A61B 18/203; H01J 61/025; H01J 65/00; H05K 7/20145; H05K 7/20154; A61B 2018/00017

(57) ABSTRACT

This application relates to a hair removal instrument, including a cooling driver, a housing, a hair removal apparatus, a cold compress part, a heat conduction part, a control board, a sealing cover, and a temperature control apparatus. The cooling driver is provided with a driving port, the cooling driver is fastened in the housing, and the housing is provided with an air outlet, a cooling channel butt-jointed to the driving port and in communication with the air outlet, and a light outlet in communication with the cooling channel. The temperature control apparatus, mounted in the housing and located on one side which is of the heat conduction part and which is away from the cooling driver, and close to the air outlet. When the temperature control apparatus detects that a temperature at the air guide hole is excessively high, the control board controls the hair removal apparatus to stop operating.

### CROSS-REFERENCE TO RELATED APPLICATIONS

The present application is a continuation-application of International (PCT) Patent Application No. PCT/CN2023/101524, filed on Jun. 20, 2023, which claims priority of the Chinese patent application No. 202221551332.5, filed on Jun. 20, 2022, the Chinese patent application No. 202221550413.3, filed on Jun. 20, 2022, and the Chinese patent application No. 202221551331.0, filed on Jun. 20, 2022, the entire contents of which are hereby incorporated by reference in their entireties.

### TECHNICAL FIELD

This application relates to the field of hair removal instrument technologies, and specifically, to a hair removal instrument.

### BACKGROUND

Photon hair removal is a new hair removal technology and a new cosmetic method on the market. Photon hair removal refers to destroying hair follicles by using a special wavelength of intense pulsed light to achieve an effect of permanent hair removal with advantages such as a fast speed, a good effect, high safety, no side effects, and painlessness. Therefore, photon hair removal is widely welcomed in the beauty and medical industries.

In an existing hair removal instrument, a turbo fan is basically used to perform air-cooled heat dissipation on heat generated inside the hair removal instrument. However, a safety hazard is easily caused when a temperature inside the hair removal instrument is excessively high.

In conclusion, how to prevent a temperature inside a hair removal instrument from being excessively high and reduce safety hazards already becomes a technical problem to be resolved urgently.

### SUMMARY

An embodiment of this application provides a hair removal instrument. The hair removal instrument includes: a cooling driver, provided with a driving port, where the cooling driver is capable of outputting a cooling medium through the driving port; a housing, where the cooling driver is fastened in the housing, and the housing is provided with an air outlet, a cooling channel butt-jointed to the driving port and in communication with the air outlet, and a light outlet in communication with the cooling channel; a hair removal apparatus, located in the housing and fastened in the cooling channel; a cold compress part, fastened on the light outlet and opposite to the hair removal apparatus, to receive light emitted by the hair removal apparatus and emit the light; a heat conduction part, fastened in the housing, where one end of the heat conduction part is located in the cooling channel, and the other end of the heat conduction part is connected to the cold compress part; a temperature control apparatus, mounted in the housing and located on one side that is of the heat conduction part and that is away from the cooling driver, and close to the air outlet; a control board, fastened in the housing, wherein all of the cooling driver, the hair removal apparatus, the cold compress part, and the temperature control apparatus are electrically connected to the control board.

In the hair removal instrument provided in this application, when the temperature control apparatus detects that a temperature at the air outlet is excessively high, the temperature control apparatus transmits information to the control board, and the control board powers off the hair removal apparatus, so that the hair removal apparatus stops operating, thereby avoiding a safety hazard caused by an excessively high temperature inside the housing.

### BRIEF DESCRIPTION OF DRAWINGS

To more clearly describe the technical solutions in the embodiments of this application, the accompanying drawings required for the descriptions of the embodiments are briefly described below. Apparently, the accompanying drawings described below are merely some embodiments of this application. A person of ordinary skill in the art may further derive other accompanying drawings based on these accompanying drawings without creative efforts.

FIG. 1 is a schematic diagram of a structure of an embodiment of a hair removal instrument according to this application;

FIG. 2 is a schematic diagram of a structure of an embodiment obtained after a power adapter and a wire are removed from a hair removal instrument according to this application;

FIG. 3 is a schematic diagram of an exploded structure of an embodiment obtained after a power adapter and a wire are removed from a hair removal instrument according to this application;

FIG. 4 is a schematic diagram of an exploded structure of another embodiment obtained after a power adapter and a wire are removed from a hair removal instrument according to this application;

FIG. 5 is a schematic diagram of an exploded structure of still another embodiment obtained after a power adapter and a wire are removed from a hair removal instrument according to this application;

FIG. 6 is a schematic diagram of an exploded structure of yet another embodiment obtained after a power adapter and a wire are removed from a hair removal instrument according to this application;

FIG. 7 is a schematic diagram of an exploded structure of yet another embodiment obtained after a power adapter and a wire are removed from a hair removal instrument according to this application;

FIG. 8 is a schematic diagram of a structure of an embodiment obtained after a bottom housing, an air guide housing, a capacitor, and a cooling driver are combined according to this application;

FIG. 9 is a schematic diagram of a structure of an embodiment obtained after a control board, an air guide housing, a heat conduction part, and a cooling driver are combined according to this application;

FIG. 10 is a schematic diagram of a structure of an embodiment obtained after a control board, a hair removal apparatus, and a cooling driver are combined according to this application;

FIG. 11 is a schematic diagram of a structure of an embodiment of a separator according to this application;

FIG. 12 is a schematic diagram of a structure of an embodiment of a cooling driver according to this application;

FIG. 13 is a schematic diagram of a structure of another embodiment obtained after a power adapter and a wire are removed from a hair removal instrument according to this application; and

FIG. 14 is a schematic diagram of an exploded structure of FIG. 13 according to this application.

### DETAILED DESCRIPTION OF THE EMBODIMENTS

The following further describes this application in detail with reference to the accompanying drawings and the embodiments. It should be particularly noted that the following embodiments are merely used for describing this application but not limiting the scope of this application. Similarly, the following embodiments are merely some rather than all of the embodiments of this application. All other embodiments derived by a person of ordinary skill in the art without creative efforts shall fall within the protection scope of this application.

Referring to the “embodiments” in this application means that a particular feature, structure, or characteristic described with reference to the embodiments may be included in at least one embodiment of this application. A person skilled in the art explicitly and implicitly understands that the embodiments described in this application may be combined with other embodiments.

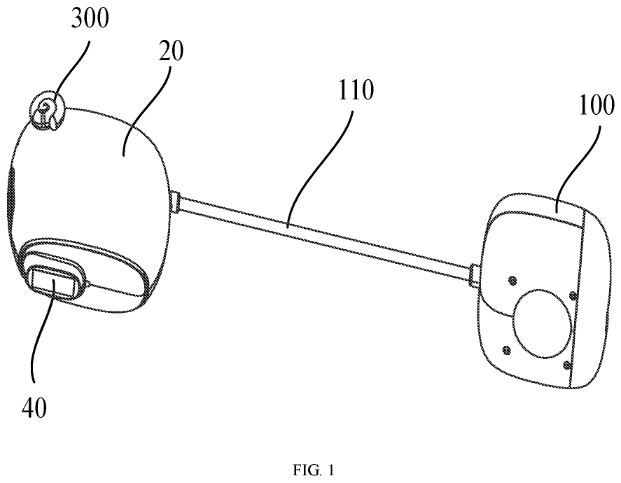

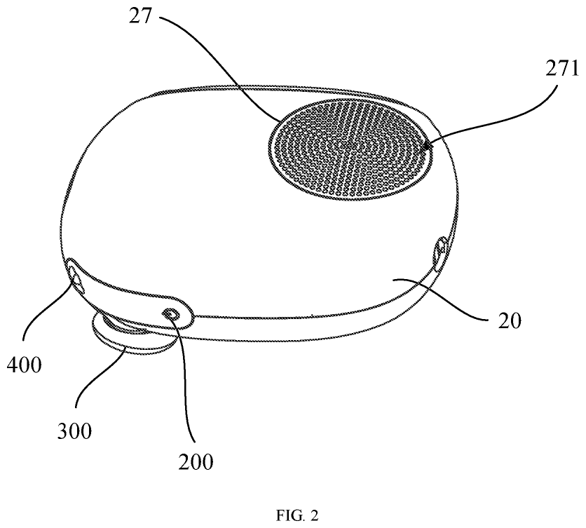

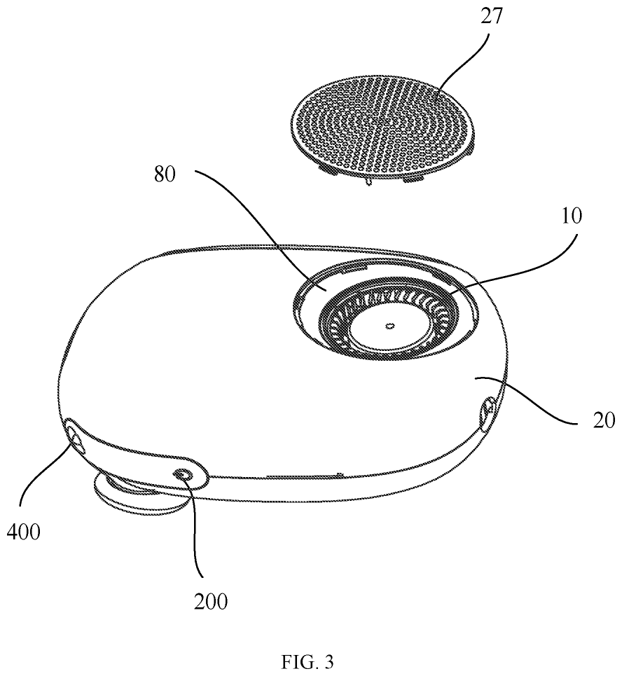

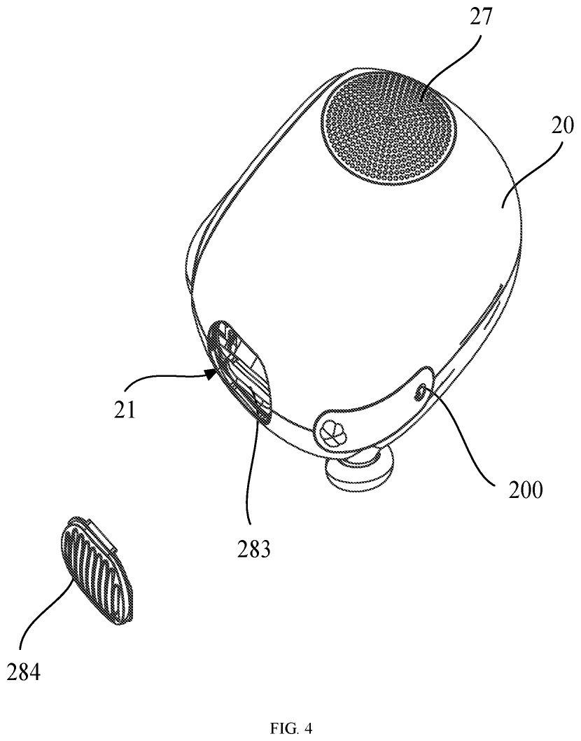

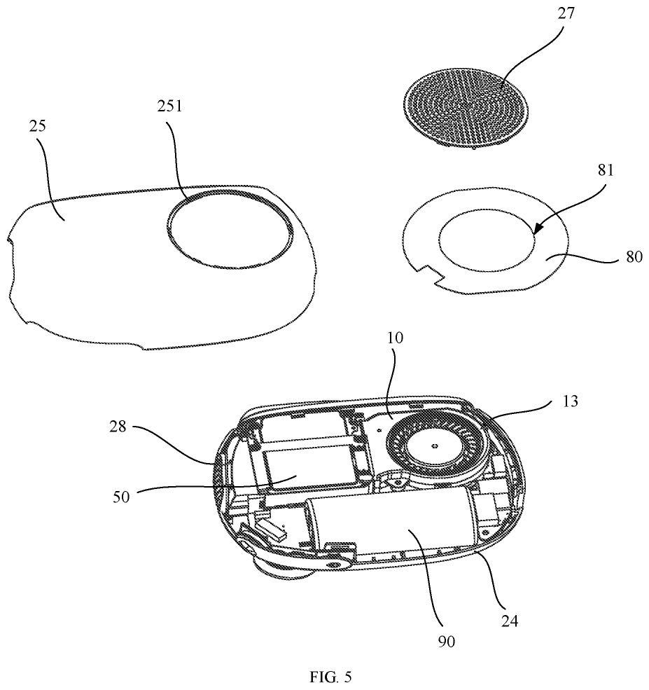

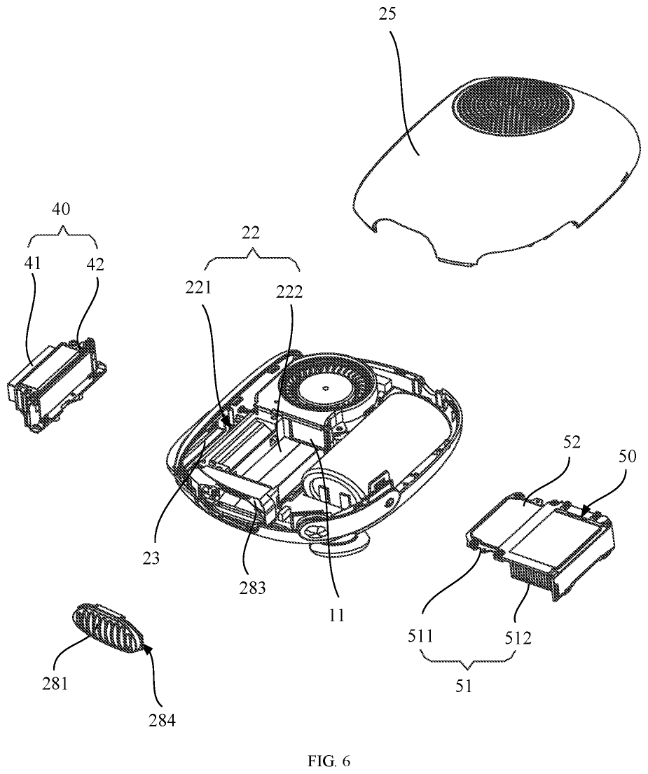

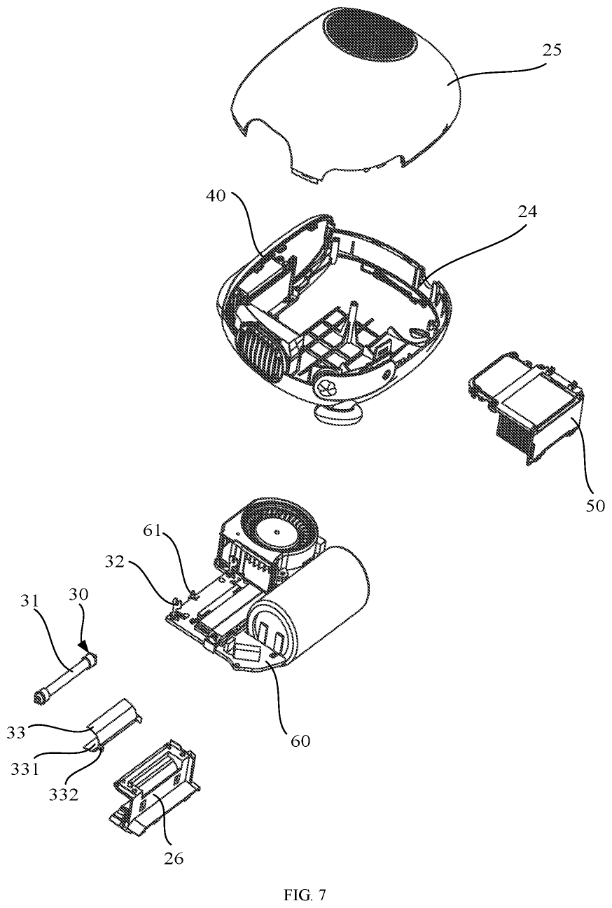

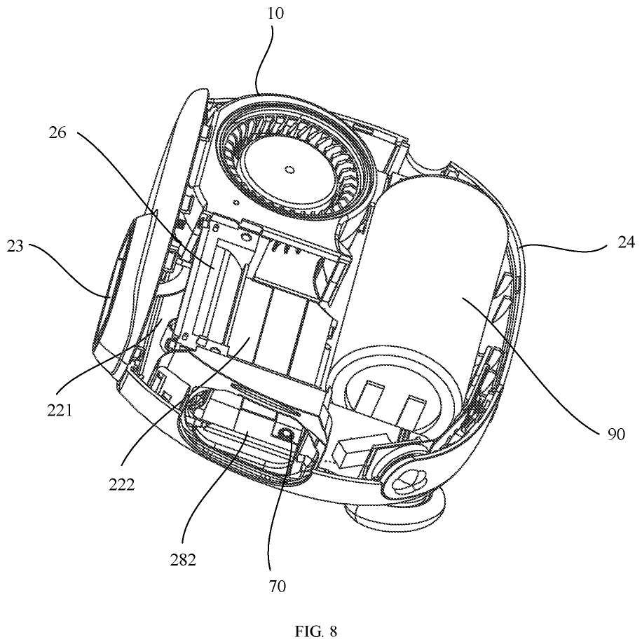

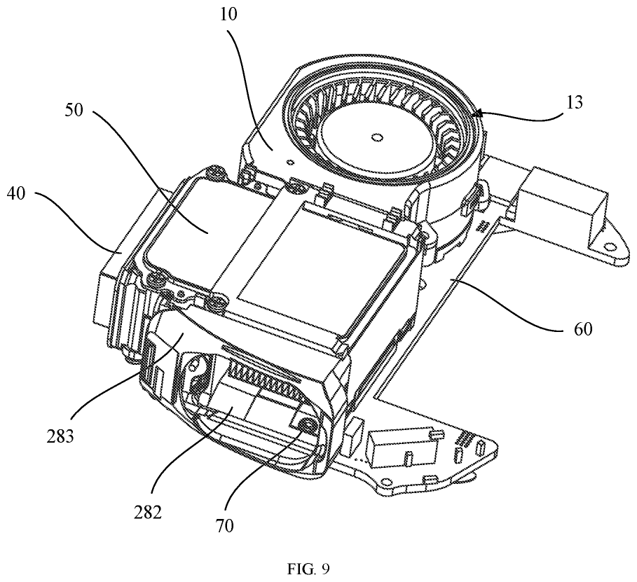

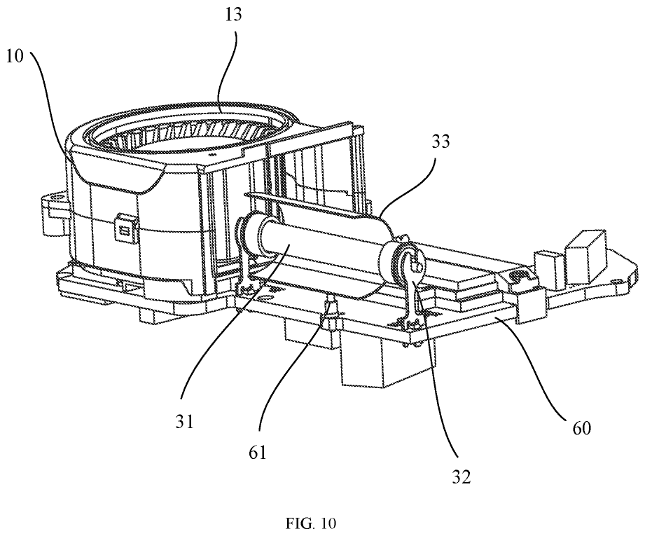

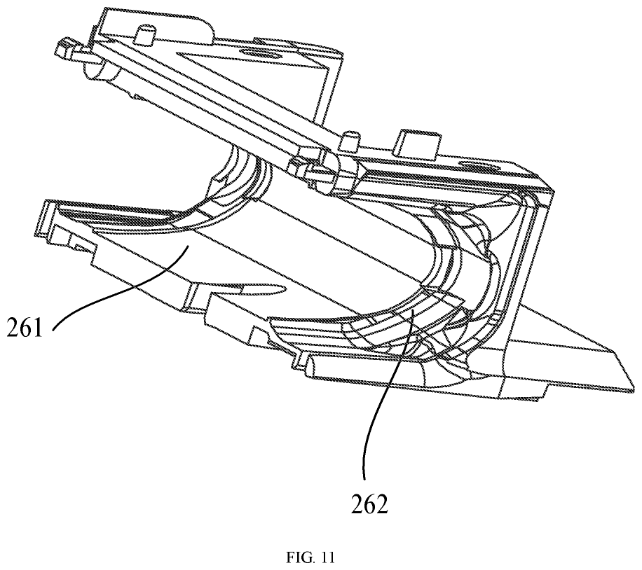

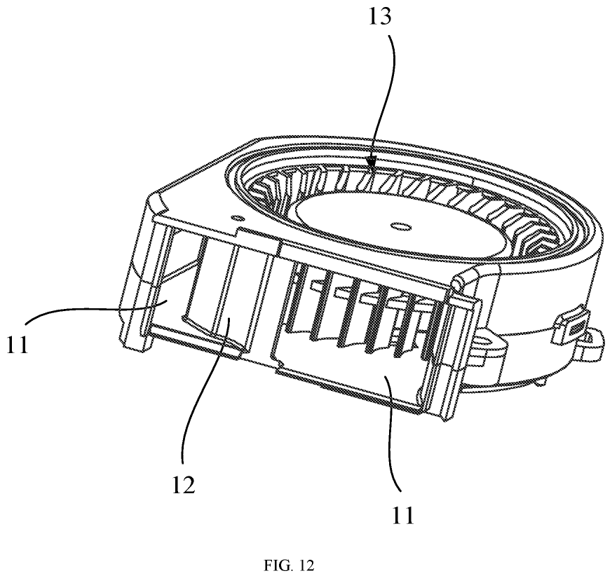

Refer to FIG. 1 to FIG. 12. An embodiment of this application discloses a hair removal instrument that can prevent an internal temperature from being excessively high. The hair removal instrument that can prevent an internal temperature from being excessively high is hereinafter referred to as a hair removal instrument for short. The hair removal instrument includes a cooling driver 10, a housing 20, a hair removal apparatus 30, a cold compress part 40, a heat conduction part 50, a control board 60, and a temperature control apparatus 70.

The cooling driver 10 is provided with a driving port 11, the cooling driver 10 may output a cooling medium through the driving port 11, and the cooling driver 10 is fastened in the housing 20. The housing 20 is provided with an air outlet 21, a cooling channel 22 butt-jointed to the driving port 11 and in communication with the air outlet 21, and a light outlet 23 in communication with the cooling channel 22. The cooling channel 22 is located at a middle part of the housing 20, the air outlet 21 and the light outlet 23 are located at edge positions of the housing 20, and the air outlet 21 and the driving port 11 are disposed opposite to each other.

The hair removal apparatus 30 is located in the housing 20 and is fastened on an inner wall of the cooling channel 22. The cold compress part 40 is fastened on the light outlet 23 and is opposite to the hair removal apparatus 30, so that the cold compress part 40 receives light, namely, laser light, emitted by the hair removal apparatus 30 and emits the light. The hair removal apparatus irradiates hair follicles with the light through skin to remove hair. The cold compress part 40 can quickly cool to relieve burning feeling caused by the light, to ensure comfort.

The cold compress part 40 may be specifically sapphire, K9 glass, crystal glass, or any light-transmission crystalline material, and may preferably be a sapphire material.

The heat conduction part 50 is fastened in the housing 20, one end of the heat conduction part 50 is located in the cooling channel 22, and the other end of the heat conduction part 50 is connected to the cold compress part 40. The heat conduction part 50 may transmit heat on the cold compress part 40 to the heat conduction part 50. The cooling medium may be used to dissipate the heat on the heat conduction part 50 from the air outlet 21, so that heat in the entire housing 20 is reduced. The control board 60 is fastened in the housing 20.

The temperature control apparatus 70 is preferably a temperature sensor, a temperature controller, or the like. The temperature control apparatus 70 is mounted in the housing 20 and is located on one side that is of the heat conduction part 50 and that is away from the cooling driver 10. All of the cooling driver 10, the hair removal apparatus 30, the cold compress part 40, and the temperature control apparatus 70 are electrically connected to the control board 60. The temperature control apparatus 70 may preferably be close to the air outlet 21. When the temperature control apparatus 70 detects that a temperature at the air outlet 21 is excessively high, the temperature control apparatus 70 transmits information to the control board 60, and the control board 60 powers off the hair removal apparatus 30, so that the hair removal apparatus 30 stops operating, thereby avoiding a safety hazard caused by an excessively high temperature inside the housing 20. For example, the temperature at the air outlet 21 is excessively high when the cold compress part 40 or the cooling driver 10 is damaged. In this case, the burning feeling caused on the skin of a human body by the laser light cannot be quickly transferred. Consequently, the skin of the human body is hurt.

In this embodiment, the hair removal apparatus 30 includes a filament-free lamp tube 31, a lamp holder 32, and a reflector cup 33 made of an electrically conductive material. Both the lamp holder 32 and the reflector cup 33 are disposed in the cooling channel 22. The lamp holder 32 is fastened on the control board 60. The reflector cup 33 is detachably mounted on the housing 20. The filament-free lamp tube 31 is mounted on the lamp holder 32 and abuts against the reflector cup 33. The reflector cup 33 is located on one side that is of the filament-free lamp tube 31 and that is away from the cold compress part 40. The reflector cup 33 reflects and focuses light of the filament-free lamp tube 31 on the cold compress part 40, and the reflector cup 33 is electrically connected to the control board 60. The lamp holder 32 provides the filament-free lamp tube 31 with a direct-current voltage. However, the filament-free lamp tube 31 does not light up in this case. The reflector cup 33 serves as a trigger electrode, and the filament-free lamp tube 31 can be triggered and controlled to light up when the control board 60 provides the filament-free lamp tube 31 with a high-voltage pulse by using the reflector cup 33.

In some embodiments, the control board 60 is welded with an elastic conductive member 61. The reflector cup 33 is fastened on an inner wall of the housing 20. The reflector cup 33 is pressed against the elastic conductive member 61. The reflector cup 33 is electrically connected to the control board 60 by using the elastic conductive member 61. The elastic conductive member 61 may be a spring sheet or an assembly including a spring. The elastic conductive member 61 has a contact pressure with the reflector cup 33 on a premise of being electrically connected to the control board 60. This ensures connection effectiveness, a solid structure, and high stability. In addition, the elastic conductive member 61 improves assembly and maintenance efficiency of the hair removal instrument.

The cooling channel 22 includes a first cooling duct 221 and a second cooling duct 222. The cooling channel 22 is a double-duct channel. The first cooling duct 221 and the second cooling duct 222 are disposed in parallel, one end of the first cooling duct 221 and one end of the second cooling duct 222 are butt-jointed to the driving port 11, and the other end of the first cooling duct 221 and the other end of the second cooling duct 222 are butt-jointed to the air outlet 21. The hair removal apparatus 30 is located in the first cooling duct 221. A part of the heat conduction part 50 is located in the second cooling duct 222. Heat dissipation of the heat conduction part 50 and heat dissipation of the hair removal apparatus 30 are separately performed, thereby effectively improving overall cooling efficiency inside the hair removal instrument. A baffle plate 12 configured to limit a capacity of a cooling medium entering the first cooling duct 221 is obliquely disposed on an inner wall of the driving port 11. The driving port 11 is butt-jointed to the first cooling channel 221 and the second cooling channel 222, so that the cooling driver 10 drives an external cooling medium to separately cool the hair removal apparatus 30 and the heat conduction part 50 through the cooling channel 22. In this way, an internal structure of the hair removal instrument is optimized, and heat dissipation efficiency inside the hair removal instrument is improved. The cooling driver 10 drives the external cooling medium to flow, so that the external cooling medium can effectively take away heat of the hair removal instrument in the double-duct channel.

The heat conduction part 50 includes a heat conduction member 51 and a graphite sheet 52 made of a graphene material. The graphite sheet 52 is fastened on the heat conduction member 51. The graphite sheet 52 made of the graphene material may enhance heat conduction efficiency of the heat conduction member 51. Heat conduction performance of the heat conduction part 50 may be significantly improved compared with that of a conventional heat conduction part. One end of the heat conduction member 51 is located in the cooling channel 22, and the other end of the heat conduction member 51 is connected to the cold compress part 40.

The heat conduction member 51 includes a vapor chamber 511 connected to the cold compress part 40 and several heat sinks 512 fastened on a bottom part of the vapor chamber 511. The graphite sheet 52 is fastened on a top part of the vapor chamber 511, the vapor chamber 511 is fastened on the inner wall of the housing 20, one end that is of the vapor chamber 511 and that is away from the cold compress part 40 covers the second cooling duct 222, the heat sinks 512 are accommodated in the second cooling duct 222, the heat sinks 512 contact a part that is of the vapor chamber 511 and that covers the second cooling duct 222, and the heat sinks 512 can improve a heat dissipation effect.

The hair removal instrument generates heat during operation. The cooling driver 10 can drive the external cooling medium to flow into the housing 20. The external cooling medium is used to take away the heat of the heat conduction part 50, to quickly cool devices inside the hair removal instrument, thereby avoiding excessive heat in an internal space of the hair removal instrument, and preventing devices in the hair removal apparatus 30 from undergoing dangerous situations such as a short circuit, burning, and explosion.

In this embodiment, the cooling driver 10 is a turbo fan, the cooling medium is preferably cooling air, the housing 20 is further provided with an air inlet 251, and the air inlet 251 is disposed corresponding to the cooling driver 10. The cooling channel 22 is located inside the housing 20, the air inlet 251 is in communication with the air outlet 21 through the driving port 11 and the cooling channel 22, and the air inlet 251 is disposed on an edge of the housing 20.

The cooling driver 10 has a guide port 13. One end of the guide port 13 is in communication with the driving port 11, and the other end of the guide port 13 is disposed corresponding to the air inlet 251 and is in communication with the air inlet 251. The hair removal instrument further includes an elastic sealing gasket 80. The elastic sealing gasket 80 has a penetrating hole 81. A gap is disposed between the housing 20 and one surface that is of the turbo fan and that faces the air inlet 251. The elastic sealing gasket 80 is clamped between the turbo fan and the housing 20 to block the gap. The air inlet 251, the penetrating hole 81, and the guide port 13 are sequentially communicated, to prevent a heat dissipation effect of the turbo fan from being poor because hot air in the housing 20 is sucked into the turbo fan again. Certainly, a situation in which hot air coming from the air outlet 21 is sucked into the cooling driver 10 again is an exception.

In some embodiments, an inner diameter of the penetrating hole 81 is greater than an inner diameter of the air inlet 251, and the air inlet 251, the penetrating hole 81, and the guide port 13 are coaxially disposed, so that external air can smoothly enter the guide port 13.

The housing 20 includes a bottom housing 24, a top housing 25 covering the bottom housing 24, and a separator 26 configured to separate the cooling channel 22 into the first cooling duct 221 and the second cooling duct 222. All of the separator 26, the cooling driver 10, the control board 60, the hair removal apparatus 30, the cold compress part 40, the heat conduction part 50, and the temperature control apparatus 70 are clamped between the bottom housing 24 and the top housing 25. The air inlet 251 is disposed corresponding to the cooling driver 10, and the air inlet 251 is in communication with the air outlet 21 through the driving port 11. The reflector cup 33 is mounted on the separator 26.

In some embodiments, the reflector cup 33 is provided with a first U-shaped groove 331 in a length direction of the reflector cup 33, and the length direction of the reflector cup 33 is consistent with a flow direction of the cooling medium. The filament-free lamp tube 31 is located in the first U-shaped groove 331 and abuts against an inner wall of the first U-shaped groove 331, so that the light of the filament-free lamp tube 31 is reflected and focused on the cold compress part 40.

The reflector cup 33 is of a U-shaped structure, and the separator 26 is provided with a second U-shaped groove 261 in a length direction of the separator 26 and several clamping grooves 262 recessed on an inner wall of the second U-shaped groove 261. The second U-shaped groove 261 is located on one side that is of the separator 26 and that faces the cold compress part 40, and the control board 60 is fastened on the bottom housing 24. Several clamping blocks 332 protrude from the reflector cup 33, the clamping blocks 332 are clamped in the clamping grooves 262, and a part of the reflector cup 33 is located in the second U-shaped groove 261, to prevent a foreign object from colliding with the reflector cup 33.

The housing 20 further includes a cover plate 27. The cover plate 27 is mounted on the housing 20 and is located at the air inlet 251, the cover plate 27 is provided with several air intake holes 271 in communication with the guide port 13, and the cover plate 27 may protect the cooling driver 10 from hurting people.

The hair removal instrument further includes a sealing cover 28. One end of the sealing cover 28 is mounted on the air outlet 21 of the housing 20 and seals the air outlet 21, and the other end of the sealing cover 28 is connected to the heat conduction part 50. The sealing cover 28 is provided with air ventilation holes 281 and an air guide hole 282. One end of the air guide hole 282 is in communication with the cooling channel 22, and the other end of the air guide hole 282 is in communication with the air ventilation holes 281. The air guide hole 282 is configured to concentrate hot air coming from the cooling channel 22, so that the hot air is blown to the air ventilation holes 281 by using the cooling medium and is dissipated from the air ventilation holes 281. In this way, the sealing cover 28 implements concentrated air exhaust.

The temperature control apparatus 70 is mounted on the sealing cover 28 and is located in the air guide hole 282. Preferably, the sealing cover 28 includes an air guide housing 283 and a sealing plate 284. The air guide housing 283 is annular, one end of the air guide housing 283 is mounted in the air outlet 21, and the other end of the air guide housing 283 is connected to the heat conduction part 50. The air guide hole 282 penetrates through the air guide housing 283. Both the sealing plate 284 and the temperature control apparatus 70 are mounted on the air guide housing 283. The sealing plate 284 is configured to seal the air guide hole 282. The air ventilation holes 281 penetrate through the sealing plate 284. The air guide housing 283 fits with the sealing plate 284 to guide the hot air out of the housing 20. This prevents hot air backflow, and can improve both heat dissipation efficiency and a heat dissipation effect. The temperature control apparatus 70 is located between the sealing plate 284 and the heat conduction part 50. The temperature control apparatus 70 is mounted on an independent sealing cover 28. The temperature control apparatus 70 is configured to detect a temperature of hot air in the air guide hole 282. When the temperature control apparatus 70 detects that the temperature at the air guide hole 282 is excessively high, the temperature control apparatus 70 transmits information to the control board 60, and the control board 60 powers off the hair removal apparatus 30, so that the hair removal apparatus 30 stops operating, thereby avoiding a safety hazard caused by an excessively high temperature inside the housing 20.

In this embodiment, the cooling channel 22 includes the first cooling duct 221 and the second cooling duct 222. The sealing cover 28 concentrates hot air coming from the first cooling duct 221 and the second cooling duct 222, and guides the hot air to the air guide hole 282 and the air ventilation holes 281, so that the blown hot air can be isolated from an inner cavity of the housing 20, thereby preventing the hot air from reentering the housing 20 after dissipation. In addition, the sealing cover 28 can prevent the temperature control apparatus 70 from collision.

The hair removal instrument further includes a capacitor 90. The capacitor 90 is fastened in the housing 20, the capacitor 90 is electrically connected to the control board 60, and the capacitor 90 may be used as a part of the control board 60 to store power.

In some embodiments, the hair removal instrument further includes a power adapter 100. The power adapter 100 is located outside the housing 20, and the power adapter 100 is connected to the control board 60 by using a wire 110, to convert an external alternating current into a direct current required by the devices inside the hair removal instrument.

The cold compress part 40 includes a cold compress plate 41 configured to adhere to skin and a refrigeration member 42 connected to the cold compress plate 41. The cold compress plate 41 is fastened on the light outlet 23 and is connected to the heat conduction part 50. Heat of the cold compress part 40 is transmitted from the cold compress plate 41 to the heat conduction part 50, so that the cold compress part 40 achieves a refrigeration effect, and a cooling rate is accelerated. The refrigeration member 42 is electrically connected to the control board 60.

The hair removal instrument further includes a start button 200 configured to control power-on of the hair removal apparatus 30. An embedding hole further penetrates through the housing 20, and the start button 200 is mounted on the embedding hole and is electrically connected to the control board 60, to control on/off of the filament-free lamp tube 31.

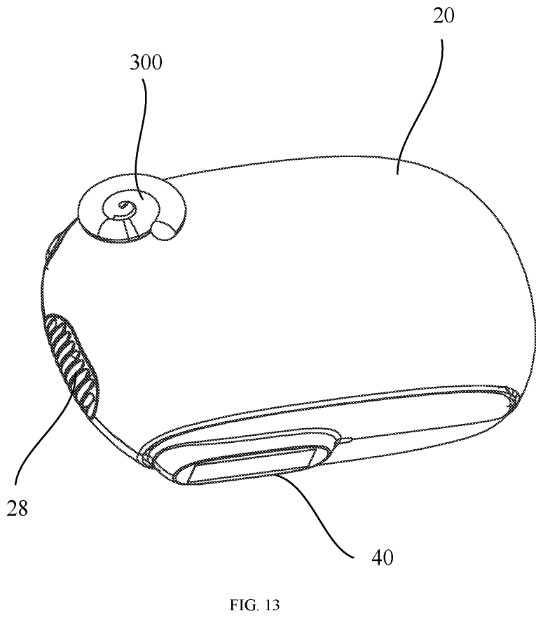

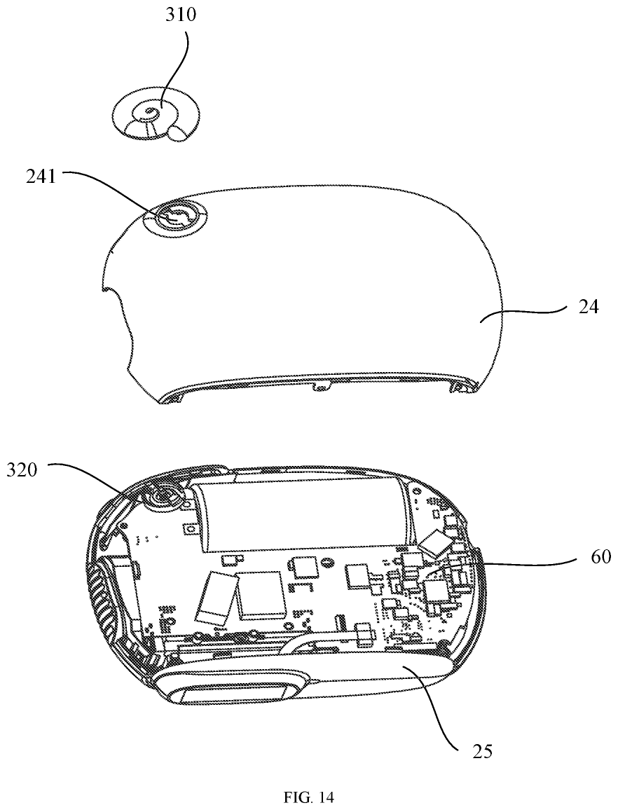

Further, refer to FIG. 13 and FIG. 14. The hair removal instrument further includes a regulator 300 configured to regulate a power of the filament-free lamp tube 31. A mounting hole 241 further penetrates through the housing 20, and the regulator 300 is mounted on the mounting hole 241 and is electrically connected to the control board 60, to regulate brightness of the filament-free lamp tube 31 at any time.

The regulator 300 includes a rotary knob 310 and a gear switch 320 mounted on the control board 60. The gear switch 320 is a rotary switch with a torsion spring inside. The rotary knob 310 is mounted on the mounting hole 241 and is rotatably connected to the gear switch 320. Power levels of the filament-free lamp tube 31 may be classified into three levels. Each time the rotary knob 310 rotates, the brightness of the filament-free lamp tube 31 changes once. The three power levels may be cycled sequentially. The rotary knob 310 returns to an initial state after each time of rotation, and the rotary knob 310 is of an ear-shaped rotary structure with a mechanical sense when being touched.

The hair removal instrument further includes a trigger pulse button 400 configured to provide the filament-free lamp tube 31 with an instantaneous high-voltage pulse. A clamping hole further penetrates through the housing 20, and the trigger pulse button 400 is mounted on the clamping hole and is electrically connected to the control board 60. The filament-free lamp tube 31 may be provided with the instantaneous high-voltage pulse when the trigger pulse button 400 is pressed. In this case, the filament-free lamp tube 31 is triggered and controlled to light up to generate the laser light, so that the hair removal instrument operates. The filament-free lamp tube 31 does not light up when the trigger pulse button 400 is not pressed.

In addition, there is a display lamp around the trigger pulse button 400 for instruction. The display lamp needs to contact the skin to be displayed. In this case, the trigger pulse button 400 can be pressed for operation. If the display lamp does not contact the skin, the display lamp is not displayed. In this case, the trigger pulse button 400 does not operate even if the trigger pulse button 400 is pressed.

Compared with the conventional technology, the hair removal instrument in this application has the following advantages:
1. In this application, the sealing cover 28 is mounted on the air outlet 21 of the housing 20, and the sealing cover 28 is configured to seal the air outlet 21. The sealing cover 28 is provided with the air ventilation holes 281 and the air guide hole 282, one end of the air guide hole 282 is in communication with the cooling channel 22, and the other end of the air guide hole 282 is in communication with the air ventilation holes 281. The air guide hole 282 is configured to concentrate the hot air coming from the cooling channel 22, so that air exhaust is performed at the air ventilation holes 281 under driving of the cooling medium. In this way, the sealing cover 28 implements concentrated air exhaust.
2. The hair removal instrument is additionally provided with the temperature control apparatus 70. The temperature control apparatus 70 is disposed in the air guide hole 282. The cooling driver 10 may output the cooling medium through the driving port 11. The cooling medium is preferably cooling air. The cooling medium may be used to dissipate the heat on the heat conduction part 50 from the air ventilation holes 281. When the temperature control apparatus 70 detects that the temperature at the air ventilation holes 281 is excessively high, the temperature control apparatus 70 transmits the information to the control board 60, and the control board 60 controls the hair removal apparatus 30 to stop operating, thereby avoiding the safety hazard caused by the excessively high temperature inside the housing 20.
3. The regulator 300 is additionally provided, and the regulator 300 may be configured to regulate the power of the filament-free lamp tube 31 to change the brightness of the filament-free lamp tube 31.
4. The graphite sheet 52 is added above the vapor chamber 511. The graphite sheet 52 made of the graphene material can enhance the heat conduction efficiency, to improve the heat dissipation effect of the heat conduction part 50.
5. The reflector cup 33 is electrically connected to the control board 60 by using the elastic conductive member 61. The elastic conductive member 61 may be a spring sheet or an assembly including a spring. The elastic conductive member 61 has the contact pressure with the reflector cup 33 on the premise of being electrically connected to the control board 60. This ensures the connection effectiveness, the solid structure, and the high stability. In addition, the elastic conductive member 61 improves the assembly and maintenance efficiency of the hair removal instrument.
6. The elastic sealing gasket 80 is additionally provided. The gap is disposed between the housing 20 and the surface that is of the turbo fan and that faces the air inlet 251. The elastic sealing gasket 80 is clamped between the turbo fan and the housing 20 to block the gap. The air inlet 251, the penetrating hole 81, and the guide port 13 are sequentially communicated, to prevent the heat dissipation effect of the turbo fan from being poor because the hot air in the housing 20 is sucked into the turbo fan again.

The foregoing descriptions are merely some embodiments of this application, and are not intended to limit the protection scope of this application. Any equivalent apparatus or equivalent process transformation that is made according to the content in the specification and the accompanying drawings of this application and that is directly or indirectly applied to another related technical field is similarly included in the patent protection scope of this application.

What is claimed is:

1. A hair removal instrument, comprising:
a cooling driver (10), provided with a driving port (11), wherein the cooling driver (10) is capable of outputting a cooling medium through the driving port (11);
a housing (20), wherein the cooling driver (10) is fastened in the housing (20), and the housing is provided with an air outlet (21), a cooling channel (22) butt-jointed to the driving port (11) and in communication with the air outlet (21), and a light outlet (23) in communication with the cooling channel (22);
a hair removal apparatus (30), located in the housing (20) and fastened in the cooling channel (22);
a cold compress part (40), fastened on the light outlet (23) and opposite to the hair removal apparatus (40), to receive light emitted by the hair removal apparatus (30) and emit the light;
a heat conduction part (50), fastened in the housing (20), wherein one end of the heat conduction part (50) is located in the cooling channel (22), and the other end of the heat conduction part (50) is connected to the cold compress part (40);
a temperature control apparatus (70), mounted in the housing (20) and located on one side which is of the heat conduction part (50) and which is away from the cooling driver (10), and close to the air outlet (21);
a control board (60), fastened in the housing (20), wherein all of the cooling driver (10), the hair removal apparatus (30), the cold compress part (40), and the temperature control apparatus (70) are electrically connected to the control board (60).

2. The hair removal instrument according to claim 1, wherein the hair removal instrument comprises a sealing cover (28), one end of the sealing cover (28) is fastened on the air outlet (21) and seals the air outlet (21), the other end of the sealing cover (28) is connected to the heat conduction part (50), the sealing cover is provided with an air guide hole (282) and several air ventilation holes (281), one end of the air guide hole (282) is in communication with the cooling channel (22), the other end of the air guide hole (282) is in communication with the air ventilation holes (281), and the air guide hole (282) is configured to concentrate hot air coming from the cooling channel (22), so that the hot air is dissipated from the air ventilation holes by using the cooling medium;
Wherein the temperature control apparatus (70) is located on an inner wall of the air guide hole (282).

3. The hair removal instrument according to claim 1, wherein the hair removal apparatus comprises a filament-free lamp tube (31), a lamp holder (32) fastened on the control board, and a reflector cup (33) made of an electrically conductive material, both the reflector cup and the lamp holder are located in the cooling channel, the reflector cup is detachably mounted on the housing and is electrically connected to the control board, the filament-free lamp tube is mounted on the lamp holder and abuts against the reflector cup, the reflector cup is located on one side that is of the filament-free lamp tube and that is away from the cold compress part, and the reflector cup reflects and focuses light of the filament-free lamp tube on the cold compress part.

4. The hair removal instrument according to claim 3, wherein an elastic conductive member (61) electrically connected to the control board is fastened on the control board, and the reflector cup is pressed against the elastic conductive member to be electrically connected to the control board by using the elastic conductive member.

5. The hair removal instrument according to claim 3, wherein the hair removal instrument further comprises a regulator (300) configured to regulate a power of the filament-free lamp tube, a mounting hole (241) penetrates through the housing (20), and the regulator is mounted on the mounting hole and is electrically connected to the control board.

6. The hair removal instrument according to claim 5, wherein the regulator comprises an ear-shaped rotary knob (310) and a gear switch (320) mounted on the control board, and the rotary knob is mounted on the mounting hole and is rotatably connected to the gear switch.

7. The hair removal instrument according to claim 3, wherein the hair removal instrument further comprises a trigger pulse button (400) configured to provide the filament-free lamp tube with an instantaneous high-voltage pulse, a clamping hole further penetrates through the housing, and the trigger pulse button is mounted on the clamping hole and is electrically connected to the control board.

8. The hair removal instrument according to claim 3, wherein the cooling channel comprises a first cooling duct (221) and a second cooling duct (222), the first cooling duct and the second cooling duct are disposed in parallel, one end of the first cooling duct and one end of the second cooling duct are butt-jointed to the driving port, the other end of the first cooling duct and the other end of the second cooling duct are butt-jointed to the air outlet, the hair removal apparatus is located in the first cooling duct, a part of the heat conduction part is located in the second cooling duct, a baffle plate (12) configured to limit a capacity of a cooling medium entering the first cooling duct is obliquely disposed in the driving port, and the cooling medium is capable of taking away heat on the heat conduction part.

9. The hair removal instrument according to claim 8, wherein the heat conduction part comprises a heat conduction member (51) and a graphite sheet (52) made of a graphene material, the graphite sheet is fastened on the heat conduction member, one end of the heat conduction member is located in the cooling channel, and the other end of the heat conduction member is connected to the cold compress part.

10. The hair removal instrument according to claim 9, wherein the heat conduction member comprises a vapor chamber (511) connected to the cold compress part and several heat sinks (512) fastened on a bottom part of the vapor chamber, the graphite sheet is fastened on a top part of the vapor chamber, the vapor chamber is fastened to an inner wall of the housing, one end that is of the vapor chamber and that is away from the cold compress part covers the second cooling duct, and the heat sinks are accommodated in the second cooling duct and contact a part that is of the vapor chamber and that covers the second cooling duct.

11. The hair removal instrument according to claim 8, wherein the housing comprises a bottom housing (24), a top housing (25) covering the bottom housing, and a separator (26) configured to separate the cooling channel into the first cooling duct and the second cooling duct, and all of the separator, the cooling driver, the control board, the hair removal apparatus, the cold compress part, the heat conduction part, and the temperature control apparatus are clamped between the bottom housing and the top housing.

12. The hair removal instrument according to claim 11, wherein the reflector cup is mounted on the separator.

13. The hair removal instrument according to claim 12, wherein the reflector cup is provided with a first U-shaped groove (331) in a length direction of the reflector cup, the length direction of the reflector cup is consistent with a flow direction of the cooling medium, the filament-free lamp tube is located in the first U-shaped groove and abuts against an inner wall of the first U-shaped groove.

14. The hair removal instrument according to claim 13, wherein the reflector cup is of a U-shaped structure, the separator (26) is provided with a second U-shaped groove (261) in a length direction of the separator and several clamping grooves (262) recessed on an inner wall of the second U-shaped groove, the second U-shaped groove is located on one side that is of the separator and that faces the cold compress part, the control board is fastened on the bottom housing, several clamping blocks protrude from the reflector cup, the clamping blocks are clamped in the clamping grooves, and the reflector cup is located in the second U-shaped groove.

15. The hair removal instrument according to claim 1, wherein the cooling driver is a turbo fan, the housing is further provided with an air inlet (251), the air inlet is disposed corresponding to the cooling driver, the cooling channel is located inside the housing, the air inlet is in communication with the air outlet through the driving port and the cooling channel, and all of the air inlet, the air outlet, and the light outlet are disposed on an edge of the housing.

16. The hair removal instrument according to claim 15, wherein the cooling driver further has a guide port (13), and the guide port is separately in communication with the air inlet and the driving port.

17. The hair removal instrument according to claim 16, wherein the hair removal instrument further comprises an elastic sealing gasket (80), the elastic sealing gasket has a penetrating hole (81), a gap is disposed between the housing and one surface that is of the cooling driver and that faces the air inlet, the elastic sealing gasket is clamped between the cooling driver and the housing to block the gap, and the air inlet, the penetrating hole, and the guide port are sequentially communicated.

18. The hair removal instrument according to claim 1, wherein the hair removal instrument further comprises a power adapter (100), the power adapter is located in the housing, and the power adapter is electrically connected to the control board by using a wire (110).

19. The hair removal instrument according to claim 1, wherein the cold compress part comprises a cold compress plate (41) configured to adhere to skin and a refrigeration member (42) connected to the cold compress plate, the cold compress plate is fastened on the light outlet and is connected to the heat conduction part, and the refrigeration member is electrically connected to the control board.

20. The hair removal instrument according to claim 1, wherein the hair removal instrument further comprises a start button (200) configured to control power-on of the hair removal apparatus, an embedding hole further penetrates through the housing, and the start button is mounted on the embedding hole and is electrically connected to the control board.
